(19) United States
(12) Patent Application Publication
(10) Pub. No.: US 2010/0055749 A1
(43) Pub. Date: Mar. 4, 2010

(54) PROCESS FOR THE PRODUCTION OF CLAVULANIC ACID

(76) Inventors: Thomas Van Der Does, Wilnis (NL); Piotr Wnukowski, Delft (NL); Bengt Olof Mathias Danielsson, Vastra Frolunda (SE); Harold Monro Moody, Gulpen (NL)

Correspondence Address: NIXON & VANDERHYE, PC, 901 NORTH GLEBE ROAD, 11TH FLOOR, ARLINGTON, VA, 22203, US

(21) Appl. No.: 12/517,139

(22) PCT Filed: Nov. 29, 2007
(86) PCT No.: PCT/EP07/62994

§ 371 Date: Nov. 5, 2009

(30) Foreign Application Priority Data
Dec. 1, 2006 (EP) 06125246.6

(51) Int. Cl. C12P 17/18

(52) U.S. Cl. 435/119

(57) ABSTRACT

The present invention relates to a process for preparation of a pharmaceutically acceptable metal salt of clavulanic acid comprising the steps of (a) fermenting of a microorganism capable of producing and excreting the clavulanic acid into the broth; (b) removing biomass and other solid material from the clavulanic acid containing fermentation broth obtained in step (a); (c) acidifying the solution obtained after step (b) to a pH between 1 and 3 and extracting the acidified solution with a partly or fully water-immiscible solvent and separating the clavulanic acid containing extract; (d) mixing the clavulanic acid containing extract obtained in step (c) with a metal donor and at least one additional cosolvent to result in an insoluble, preferably crystalline metal clavulanate salt with a yield of at least 80%; and (e) separating the insoluble, preferably crystalline, metal clavulanate salt from the mixture obtained in step (d).

The present invention relates to a novel process for the production of clavulanic acids and pharmaceutically acceptable salts thereof.

Clavulanic acid is the common name for (2R,5R,Z)-30(2 hydroxyethylidene)-7-oxo-4-oxa-l-azabicyclo [3.2.0]heptane-2-carboxylic acid. Clavulanic acid and its alkali metal salts and esters are active as inhibitors of beta-lactamase produced by some Gram (+) as well as Gram (−) micro-organisms. Suitable microorganisms that are capable of producing clavulanic acid as well as suitable conditions for culturing these microorganisms are well known in the art. For example strains belonging to the genus Streptomyces such as S. clavuligerus NRRL 3585 (U.S. Pat. No. 4,110,165), S. jumonjinensis NRRL 5741 (British Patent 1,563,103), S. katsurahamanus IFO 13716 (Japanese Patent 83,009,579) and Streptomyces sp.P6621FERM 2804 (Japanese Patent 55,162,993) are used. For the preparation of clavulanic acid by fermentation process Streptomyces clavuligerus is preferred. The recovery and purification of clavulanic acid requires as a first step removal of the biomass and other solid material from the fermentation broth. This may be carried out according to methods well known in the art. For example, flocculants may be added followed by filtration, e.g. vacuum filtration may be used. Alternatively, microfiltration may be used as described in WO96/28452. In most production processes, the clavulanic acid extracted into a partly- or fully water immiscible solvent (e.g. ethyl acetate). This step involves acidification of the filtered clavulanic acid containing solution to a pH between 1 and 3 after which the undissociated, protonated clavulanic acid can be extracted into an organic solvent. In most if not all industrial processes, ethyl acetate is used as the organic solvent for extraction.

In order to obtain a clavulanic acid product of high purity and quality, all current industrial production processes use an intermediate crystallization step of an amine salt of clavulanic acid. EP-A-0026044 discloses the use of the tertiary butylamine salt of clavulanic acid as an intermediate for purification of clavulanic acid. Tertiary butylamine, however, is a toxic compound and is also difficult to remove from wastewater giving rise to serious pollution concerns. EP-A-0562583 discloses use of salts of clavulanic acid with N,N′-monosubstituted symmetric ethylene diamines such as N,N′-diisopropylethylene diammonium diclavulanate as useful intermediates for isolation and preparation of pure clavulanic acid or alkaline metal clavulanate salts from ethyl acetate extract. WO93/25557 discloses numerous amines as intermediates for preparation of clavulanic acid or pharmaceutically acceptable salts or esters. EP-A-0594099 discloses use of tertiary octylamine with clavulanic acid as an intermediate in preparation of clavulanic acid or pharmaceutically acceptable salts. WO94/21647 discloses use of N,N′-substituted diamines such as N,N′-diisopropylethylene diammonium diclavulanate as a useful intermediate for preparation of clavulanic acid and alkali salts. WO94/22873 discloses use of novel tertiary diammonium salts of clavulanic acid such as N,N,N′,N′-tetramethyl-l,2-diaminoethane clavulanate as a useful intermediate for preparation of clavulanic acid and salts thereof.

The use of the amine salt of clavulanic acid as an intermediate has the advantage that a final product of high purity is obtained; the disadvantages however are that the production process contains one or more additional steps, which result in an overall decreased yield and higher cost price. Also, the amines used are expensive which is again reflected in the cost price. Furthermore, the amines used are toxic which necessitates special operational requirements while also the waste stream of such production plants contains these toxic amines.

Due to the disadvantages in relation to the use of the amines, several companies developed a production process for clavulanic acid, which no longer requires the use of such amines. Examples of these processes have been disclosed in WO95/21173, WO95/34194, WO96/28452, WO97/05142 and WO98/42858. WO95/21173 relates to a process for the preparation of a salt of clavulanic acid, wherein clavulanic acid in solution in a wholly or partly water-immiscible organic solvent is contacted in a region of high turbulence and/or shear stress, with a salt precursor compound of a salt forming cation with a counter anion in solution or suspension, the counter anion being capable of exchange with clavulanate anion, in the presence of water, such that a solution of the salt of clavulanic acid in an aqueous phase is formed, then the organic solvent and aqueous phases are physically separated during a separation step, followed by a further processing step in which the said salt of clavulanic acid is isolated from solution as a solid. WO95/34194 relates to a process for manufacturing an alkali metal salt of clavulanic acid wherein impure clavulanic acid in aqueous solution is extracted by a solvent mixture of a ketone and alkyl acetate under acidic condition, and a solution of alkali metal salt of alkanoic acid dissolved in ketone or alkanol solvent is added thereto to obtain pure alkali metal salt of clavulanic acid. WO96/28452 relates to a process for preparation of a pharmaceutically acceptable salt of clavulanic acid comprising the steps of: removing solids from a clavulanic acid containing fermentation broth by microfiltration; acidifying the filtrate to a pH between 1 and 3; extracting the acidified filtrate with a water immiscible solvent and separating the clavulanic acid containing extract; mixing the extract with a metal donor and at least one additional solvent and separating the metal clavulanate salt from the solution. WO97/05142 relates to a process for the preparation of pharmaceutically acceptable quality potassium clavulanate by the direct precipitation of clavulanic acid as the potassium salt, which has not been pre-purified by the formation of an intermediate amine salt whereby the subject matter of WO95/21173 is excluded. WO98/42858 relates to a process for the isolation of a pharmaceutically acceptable alkali metal salt of clavulanic acid from a fermentation broth containing impure clavulanic acid comprising the steps of filtration of the fermented broth, extraction of the clavulanic acid to a water immiscible or partly water immiscible solvent at pH from 1.2-2, precipitation of an alkali metal salt A of clavulanic acid by addition of a solution of an alkali metal alkylalkanoate, characterized by the following steps: before the filtration the fermented broth containing clavulanic acid is diluted with water, a flocculating agent is added and the pH is adjusted to 3-5. For further purification the alkali metal salt A of clavulanic acid is converted to clavulanic acid by addition of an inorganic acid and is extracted into a water immiscible or partly water immiscible solvent; a solution of a different alkali metal B alkyl alkanoate is added and the alkali metal salt B of clavulanic acid is precipitated.

Each of the amine-free processes described hereinbefore, has one or more disadvantages, which have prohibited the implementation in industrial production processes until the present day (i.e. 8-10 years after the publication thereof). A major disadvantage of the known amine free processes is that under applied process conditions to give economically acceptable yields, the alkali metal salts of clavulanic acid are not pure enough for pharmaceutical use. The quality of these end products may be improved but only at the expense of the overall yield, in particular the yield of the crystallization process, which again for economic reasons, prohibits the industrial application of these amine free prior art processes. Therefore, there is still an urgent need for an efficient, amine-free and cost effective production process for clavulanic acid. It is an object of the present invention to provide such an efficient, amine-free and cost effective production process for clavulanic acid wherein the clavulanic acid and its pharmaceutically acceptable salts are obtained in a high yield and of high purity while avoiding the use of toxic amines or lithium compounds.

In a first aspect the invention provides a process for the preparation of a pharmaceutically acceptable metal salt of clavulanic acid comprising the steps of (a) fermenting of a micro-organism capable of producing clavulanic acid and excreting it into the broth; (b) removing biomass and other solid material from the clavulanic acid containing fermentation broth obtained in step (a); (c) acidifying the solution obtained after step (b) to a pH between 1 and 3 and extracting the acidified solution with a partly or fully water-immiscible solvent and separating the clavulanic acid containing extract and optionally concentrating and/or decolouring by carbon treatment the extract thus obtained; (d) mixing the clavulanic acid containing extract obtained in step (c) with a metal donor and at least one additional cosolvent to result in an insoluble, preferably crystalline metal clavulanate salt with a yield of at least 80%; and (e) separating the insoluble, preferably crystalline, metal clavulanate salt from the mixture obtained in step (d).

As described herein before, suitable microorganisms that are capable of producing clavulanic acid as well as suitable conditions for culturing these microorganisms are well known in the art. For example strains belonging to the genus Streptomyces such as S. clavuligerus NRRL 3585 (U.S. Pat. No. 4,110,165), S. jumonjinensis NRRL 5741 (British Patent 1,563,103), S. katsurahamanus IFO 13716 (Japanese Patent 83,009,579) and Streptomyces sp.P6621FERM 2804 (Japanese Patent 55,162,993) are used. For the preparation of clavulanic acid by fermentation process Streptomyces clavuligerus is preferred.

The removal of the biomass and other solid material from the fermentation broth may be carried out according to any suitable method known in the art. For example, in order to facilitate facile filtration e.g. vacuum filtration, of biomass and other solid material, flocculants, acetone and/or filter aid may be added to the broth. In the case of using acetone, the latter may be removed from the filtrate by evaporation, preferably in vacuum. Alternatively, microfiltration of the whole broth may be used to remove biomass and other solid material as described in WO96/28452.

Acidification of the, biomass and other solid material free, solution of clavulanic acid may be carried using acids known in the art. The final pH is preferably such that the undissociated clavulanic acid can be extracted efficiently into the partly or fully water-immiscible organic solvent. Preferably said pH is between 1 and 3. Examples of suitable organic solvent that are partly or fully water-immiscible are alkyl acetates such as ethyl acetate or ketones such as methyl-iso-butylketone. Recovery of the organic phase containing the clavulanic acid may be carried out according to methods known in the art thus yielding what will be defined here as the “extract”. At this stage, several additional process steps may be applied. For instance, coloured contaminants may be removed from the “extract” by a carbon treatment, such as an activated carbon treatment according to methods known in the art, for instance using the advanced process as described in WO2005/039756. Furthermore, it may be necessary to concentrate the “extract” in case the concentration of clavulanic acid in the “extract” is too low to give satisfactory yields and product quality after further processing. A suitable way of concentrating the “extract” is by evaporation under vacuum conditions. This concentration step may be carried out before as well as after the carbon treatment or both.

The “extract”, optionally decoloured and/or concentrated, is then mixed with a metal donor and at least one additional cosolvent. Preferred metal donors are compounds that donate metal ions, preferably the alkali metals sodium (Na), potassium (K) and lithium (Li). Preferably, the metal donor donates sodium (Na) or potassium (K), highly preferred is potassium (K). Suitable metal donors are salts of the metals mentioned before, preferably organic salts, more preferably organic acid salts such as methyl salts of acetic acid or 2-ethylhexanoic acid, most preferred is a metal salt of 2-ethylhexanoic acid. The most preferred metal donor of the present invention is potassium 2-ethylhexanoate.

Preferably, the molar ratio of the clavulanic acid versus the metal donor during the mixing is kept equal to or less than 1, more preferably equal to or less than 0.9, more preferably equal to or less than 0.8 and most preferably equal to or less than 0.7.

The clavulanic acid containing “extract” which is mixed with the metal donor preferably has a concentration of clavulanic acid which allows a yield of at least 80% in the precipitation/crystallization step, preferably the concentration of clavulanic acid in the “extract” is at least 35 g/L (measured as clavulanic acid), more preferably at least 37 g/L, more preferably at least 40 g/L, more preferably at least 43 g/L, more preferably at least 46 g/L more preferably at least 50 g/L. The concentration of clavulanic acid in the “extract” is preferably less than 100 g/l, more preferably less than 90 g/L more preferably less than 80 g/L and most preferably equal or less than 70 g/l.

In addition, the amount of coloured contaminants in the clavulanic acid containing “extract” which is mixed with the metal donor preferably is low. Preferably the colour value, expressed as the “Hunter b” value is 8 or lower, more preferably below 7, more preferably below 6, more preferably below 5, more preferably below 4 and most preferably below 3. The purity and quality of the final metal clavulanate salt crystals, is affected by the coloured contaminants in the “extract”. This means that with lower concentrations of coloured contaminants in the “extract give a higher purity and quality of the final metal clavulanate salt crystals.

According to the process of the present invention, the metal clavulanate salt obtained after mixing the “extract” with the metal donor and at least one cosolvent, is insoluble in the mixture thus obtained. The insoluble metal clavulanate salt may form a precipitate, but, in a preferred embodiment of the invention, the insoluble metal clavulanate salt forms crystals. The crystals may be of different forms such as needles and blocks.

In the process according to the present invention, the yield of the formation of the insoluble, preferably crystalline metal clavulanate salt is at least 80%. Preferably the yield is at least 82%, more preferably at least 84%, more preferably at least 88%, more preferably at least 92%, more preferably at least 95% and more preferably at least 98%. Most preferably the yield of the crystallization process is 100%. The yield of the formation of the insoluble, preferably crystalline metal clavulanate salt is defined herein as the relative amount of clavulanic acid in the metal clavulanate salt precipitate or crystals versus the amount of clavulanic acid in the clavulanic acid containing extract (taken as 100%) and is expressed as a percentage value. The yield of the crystallization process is positively influenced by the concentration of clavulanic acid in the “extract”. This means that at higher concentrations of clavulanic acid in the “extract”, higher crystallization yields are obtained. For instance, at a concentration of clavulanic acid in the “extract” of at least 35 g/L the yield of the crystallization process has been found to be at least 80%.

The metal donor may be added as a solution or suspension in any suitable solvent system. For example, the metal donor may be added as a solution in ethyl acetate. In one embodiment of the present invention, the cosolvent is added separately to the crystallization mixture. The metal donor may also be added as a solution or suspension in the cosolvent.

Preferably, the cosolvent is an alcohol, such as methanol, ethanol and isopropanol. Most preferred is ethanol. Optionally, the cosolvent additionally contains some water, preferably between 0 and 10% (v/v), preferably between 1 and 9% (v/v), preferably between 2 and 8% (v/v), most preferably between 5 and 7%. In a preferred embodiment of the present invention, the metal donor potassium 2-ethylhexanoate is added as a solution in the cosolvent ethanol, preferably ethanol containing some water as described hereinbefore. Most preferred is a solution of potassium 2-ethylhexanoate in ethanol containing 5% water (v/v). The concentration of the metal donor preferably is between 0.1 and 2.0 M, more preferably between 0.25 and 1.0 M and more preferably between 0.4 and 0.6 M, most preferably around 0.5 M.

The mixing of the clavulanic acid containing “extract” with the metal donor may be carried out in the following ways: mixing instantaneously the total volume of clavulanic acid containing “extract” with the total volume of the metal donor, or, more preferably, adding the metal donor slowly to the total volume of the clavulanic acid containing “extract” such that the molar ratio of metal donor versus clavulanic acid initially is very low and gradually rises to a value of around 1, or, more preferably, in the reverse way, adding the clavulanic acid containing “extract” slowly to the total volume of the metal donor such that the molar ratio of the clavulanic acid versus the metal donor initially is very low and gradually rises to a value of around 1, or, most preferably in a way wherein the clavulanic acid containing “extract” and a solution of the metal donor in cosolvent, are added simultaneously to a vessel. In order to facilitate mixing at the start of any of the procedures mentioned, the vessel may contain solvent used for extraction or cosolvent or a combination thereof.

Slow addition means that the metal donor or the clavulanic acid containing “extract” is added to the other solution during a time period of 20-100 minutes, preferably of 30-90 minutes, most preferably of 40-80 minutes. These additions may be carried out at temperatures between 0 and 40° C., preferably between 10 and 35° C., more preferably between 20 and 30° C.

After completion of the additions, the resulting mixture is cooled down to a lower temperature, preferably between 0 and 15° C., more preferably between 0 and 10° C., most preferably between 0 and 5° C. A highly preferred temperature is between 3.5 and 4.5° C. At these lower temperatures, the crystallization of the metal-clavulanate is promoted. The crystals of the metal clavulanate, preferably potassium clavulanate may be separated and processed further into a dry product according to methods known in the art.

### Materials and Methods

Extracts of clavulanic acid can be characterized by the Hunter colour parameter, in particular the Hunter b value. The Hunter b value of an extract is measured in a Colorimeter, Minolta CT210 in a cuvet of 1 cm diameter—for details on the method see Publication number 9242-4830-92 of MINOLTA Co Ltd, 1998. The equipment is calibrated with distilled water. Extracts (either before or after treatment with carbon) are measured as such.

### EXAMPLES

### Example 1

500 ml of a solution containing 44.6 g/l of clavulanic acid in dry ethyl acetate was obtained after carbon treatment as described in WO2005/039756. The carbon treated extract (CTE) with a colour value Hunter b=4.6 was added in the course of 45 min to 316 ml of a 0.5 M solution the potassium salt of 2-ethylhexanoic acid in ethanol/water 95/5 (v/v) at 30° C. The mixture was stirred for an additional 60 min at 4° C. The crystals were filtered and washed with 2*50 ml of acetone. The white crystals were dried in vacuum at room temperature. The yield was 84%.

### Example 2

A crystallization vessel was pre-charged with 48 ml ethanol, 77 ml ethyl acetate and 0.5 g seed crystals (potassium clavulanate) that were produced according to method of example 1. Then, 500 ml of a solution of clavulanic acid in dry ethyl acetate obtained as described in WO2005/039756 containing 37.5 g/l clavulanic acid, colour Hunter b=3.95 was added in the course of 80 min. Simultaneously, 309 ml of 0.5 M solution the potassium salt of 2-ethylhexanoic acid in ethanol/water 95/5 (v/v) was added. The temperature during addition was 20° C. The mixture was stirred for an additional 60 min at 4° C. The crystals were filtered and washed two times with 50 ml of acetone. The white crystals were dried in vacuum at room temperature. The yield was 85%.

### Example 3

A 1 litre crystallization vessel was pre-charged with 300 ml ethanol containing 5% water, 500 ml ethyl acetate and 22.08 g seed crystals (potassium clavulanate). Subsequently, simultaneously 1100 ml of carbon treated extract containing 44.6 g/l of clavulanic acid with a colour value Hunter b=3.56 and 700 ml of a 0.5 M solution of the potassium salt of 2-ethylhexanoic acid KEH) in ethanol/water 95/5 (v/v) were added. The temperature was 30° C. in the reactor and the stirring speed was 260 rpm. Total addition time was 134 min (1800/134=13.4 ml/min).

At the same time product was removed from the crystallization vessel at the same flow rate as the addition of the clavulanic acid and KEH (i.e. 13.4 ml/min), thereby maintaining a constant volume in the crystallization vessel and the product was collected in a collecting vessel. The temperature in the collecting vessel was 20° C.

The crystals in the collecting vessel were filtered and washed 2 times with 100 ml acetone. The white crystals were dried in vacuum at room temperature and had a Hunter b value of 0.70. The yield was 81%.

1. A process for preparation of a pharmaceutically acceptable metal salt of clavulanic acid comprising the steps of
(a) fermenting of a microorganism capable of producing and excreting the clavulanic acid into the broth;
(b) removing biomass and other solid material from the clavulanic acid containing fermentation broth obtained in step (a);
(c) acidifying the solution obtained after step (b) to a pH between 1 and 3 and extracting the acidified solution with a partly or fully water-immiscible solvent and separating the clavulanic acid containing extract;
(d) mixing the clavulanic acid containing extract obtained in step (c) with a metal donor and at least one additional cosolvent to result in an insoluble, preferably crystalline metal clavulanate salt with a yield of at least 80%; and
(e) separating the insoluble, preferably crystalline, metal clavulanate salt from the mixture obtained in step (d).

2. A process according to claim 1 wherein the clavulanic acid containing extract used in step (d) contains at least 35 gram clavulanic acid per liter extract.

3. A process according to anyone of claim 1 wherein the molar ratio of the clavulanic acid versus the metal donor during the mixing in step (d) is kept equal to or less than 1.

4. A process according to claim 1 wherein the clavulanic acid containing extract obtained after step (c) is treated with carbon whereby the carbon treated extract has a colour value equal to or lower than 8.

5. A process according to claim 1 wherein the additional cosolvent of step (d) is ethanol preferably a mixture of ethanol and water.

6. A process according to claim 1 wherein the pharmaceutically acceptable metal salt of clavulanic acid is the potassium or the sodium salt, preferably the potassium salt.
